(19) United States
(12) Patent Application Publication
(10) Pub. No.: US 2015/0157493 A1
(43) Pub. Date: Jun. 11, 2015

(54) Genital Shield for Reducing the Transmission of Sexually Transmitted Diseases

(71) Applicant: Arif H. Agha, MD, Oakbrook, IL (US)

(72) Inventor: Arif H. Agha, MD, Oakbrook, IL (US)

(21) Appl. No.: 14/100,318

(22) Filed: Dec. 9, 2013

(51) Int. Cl. A61F 6/06; A61F 6/02

(52) U.S. Cl. CPC A61F 6/06; A61F 6/02

(57) ABSTRACT

This invention relates to a genital shield capable of being coupled with a condom for use in the genito-scrotal and suprapubic area of a male and/or the perineal or labial area of a female. This shield comprises an adhesive dressing for application on a user's genital region that is bio-occlusive, thin, flexible, unobtrusive to the senses, and aesthetically pleasing to a user.

### CROSS-REFERENCE TO RELATED APPLICATIONS

Not Applicable.

### STATEMENT REGARDING FEDERALLY SPONSORED RESEARCH OR DEVELOPMENT

Not applicable.

### REFERENCE TO SEQUENCE LISTING, A TABLE, OR A COMPUTER PROGRAM LIST COMPACT DISC APPENDIX

Not applicable.

### BACKGROUND OF THE INVENTION

Well before current modern times, people have endeavored to prevent pregnancy and disease with various forms of barrier type devices that shielded one's genitalia from contact with a partner's. Today, common, effective barrier devices include such devices as the male or female condoms and dental dams. Male condoms, in particular, are a popular form of using a barrier to prevent the spread of disease and to reduce the rate of an unwanted pregnancy. Users have largely found condoms to be unobtrusive in terms of sensation or aesthetic appeal which adds to their popularity. While barrier devices have been successful in reducing disease and pregnancy, there is still a risk of spreading disease as condoms or other barrier devices do not cover all of a person's possible infected area. This is especially true of diseases such as genital herpes, genital warts (Human Papilloma Virus; “HPV”), syphilis, chanchroid, and other diseases that are spread through skin to skin contact and can be found on the genito-scrotal and suprapubic area of a male and/or the genital and perineal area of a female. As such there have been many attempts to cover this area of the body to help reduce the spread of disease.

One known solution to this problem is seen in U.S. Pat. No. 4,834,114 (Boarman) issued on May 30, 1989 and U.S. Pat. No. 5,113,873 (Boarman) issued on May 19, 1992. This relates to a contraceptive system similar in design to the male condom but including a genital shield member. A releasable fastener mechanism secures the shield to the individual. This invention is for use by females as the condom like portion is inserted into the vagina and the shield is releasably attached to the pubic area of the female. This problem with these known solutions is that the releasable fastener mechanism tends to have poor adherence qualities which results in lowered protection against disease transmission.

Another known solution is seen in U.S. Pat. No. 5,413,117 (Wills) issued on May 9, 1995. This invention relates to a prophylactic device having a shield member that lies over the pubic region. This device is held onto a user's body by adjustable straps. One known problem with this solution is that the adjustable straps of this invention are not viewed positively by users as there is friction and movement with the straps and the straps are not aesthetically pleasing.

Another known solution is seen in U.S. Pat. No. 5,168,881 (Reddy) issued on Dec. 8, 1992. This relates to a prophylactic device which is intended to be worn as a garment and further is able to provide a shield for either a male or female perineum for added protection against disease. This device also utilizes straps for holding the device in place which causes friction and unwelcome movement of the device and the garment can be unappealing to a user.

Another solution is seen in U.S Pub. No. 20100071702 (Stuhrlingh). This relates to a condom and molded shield portion that rests against a user's pubic and scrotum area. A ring retains the condom portion over the penis. An issue with this type of solution is that a molded portion rarely fits all users as in order to be effective, the mold would have to properly fit against a user's body. Since bodies vary greatly in size, the molding would not fit all body types properly and would need to be custom made. Further, moldings tend to be semi-rigid or hard by nature and this would significantly reduce sensation during intercourse for a user.

All of the above known prior art solutions attempted to solve the issue of creating an effective barrier against disease but none were able to do it in an unobtrusive, aesthetically pleasing and bio-occlusive manner that appropriately fit a user without unintended movement. The issue with any device providing a barrier in the genital area is that this area of the body has non-conforming anatomy to such known shields. Additionally, there are issues of moisture, poor adherence against the skin, displacement due to friction or moisture, and decreased sensation in users. Further, many of the known solutions require that the proscribed condom be used with the devices instead of allowing a user preference in condom choice which is known to be an important feature in the popularity of barrier shields. This invention solves all of these problems by utilizing an adhesive film dressing as a shield that is known to adhere well to skin, does not diminish sensation or aesthetic appeal, is flexible enough to adhere to any body type and surface, and allows a user to use his/her preferred condom in conjunction with the shield.

The above references to and descriptions of prior proposals or products are not intended to be, and are not to be construed as, statements or admissions of common general knowledge in the art. In particular, the above prior art discussion does not relate to what is commonly or well known by the person skilled in the art, but assists in the understanding of the inventive step of the present invention of which the identification of pertinent prior art proposals is but one part.

### BRIEF SUMMARY OF THE INVENTION

This invention relates to a genital shield for use in conjunction with a condom to aid in reducing the spread of sexually transmitted disease. More specifically it relates to a shield comprising a bio-occlusive, aesthetically pleasing barrier that does not reduce sensation that is applied to the genital region where a condom does not cover. The barrier comprises a dressing having an adhesive and non-adhesive side wherein the dressing is coupled to a backing sheet on the adhesive side and a border on the non-adhesive side.

In its preferred embodiment, the dressing comprises a thin, flexible, bio-occlusive material. Since this dressing will be used by persons engaging in sexual intercourse, a preferred dressing must also be aesthetically pleasing to all of a user's senses. The dressing must be thin enough so as to not interfere with sensations on the skin. A preferred material will also be transparent; however one skilled in the art will recognize that translucent, semi-transparent, or solid materials may be anticipated. In this type of use, the more transparent a dressing is the more aesthetically pleasing it may be considered.

Such preferred dressing material may include material such as transparent film dressings commonly used in wound care or to dress surgical sites. There are a wide variety of dressing materials marketed under a variety of names such as Tegaderm® (3M), Bioclusive® (Johnson & Johnson Company), Hydrofilm® (Paul Hartmann Aktiengesellschaft), Opsite® (T.J. Smith & Nephew), Suresite® (Medline Industries), or Mepore® (Molnlycke Health Care AB Corporation). This list is not intended to be inclusive of the types of surgical wound dressings available and only serves to list examples of possible known dressings that hold characteristics that would be desirable to be used in the dressings of this invention. These film dressings are known to be highly effective in preventing wound or surgical infection in patients while giving patients a thin, flexible, unobtrusive and transparent dressing. Patients are able to wear these dressings for an extended period of time without the dressing separating from the skin and allowing for a user to continue normal activities without significant restriction. The dressings does not allow bacteria or viruses to be introduced to the skin area it is covered when used properly. Such material would translate well into the material for this dressing as the material provides a fluid-proof bacterial or viral barrier against the skin. The thin, transparent, flexible, bio-occlusive nature of this material makes it a preferred choice of material for the dressing of this invention.

The dressings are contoured to fit male or female anatomy. With the male anatomy contouring version of the dressing, two components may be anticipated to be used in conjunction with each other. There is a dressing contoured to fit the scrotal area of the male anatomy and a dressing contoured to fit the male suprapubic area of the male anatomy. It is anticipated that the dressing contoured to fit the male scrotal region will wrap around the male scrotum and the undersurface (ventral aspect) of base of the penis. The suprapubic dressing component is anticipated to cover the male genital (pubic hair area) and the adjoining skin as well as the upper (dorsal) surface of the base of the penis. The two male dressing components, when applied appropriately, will overlap each other where the two parts meet so that no skin is left uncovered. An extended version of the suprapubic dressing may cover the groin fold. With the female anatomy contouring version of the dressings, there is a dressing contoured to fit the mons pubis and perineal region of the female anatomy. An extended version may have dressing lips to cover the labial skin. Both female dressing versions have a central aperture for introitus. One skilled in the art will recognize that the dressing may be in a variety of sizes to meet the needs of a population with a variety of body sizes.

The dressing further comprises an adhesive side coupled to a backing sheet. The adhesive side of the dressing is to be applied to the body after it is peeled away from the backing sheet. The backing sheet is preferred to be a stiff sheet of paper, plastic, or other suitable material. The dressing is peeled away from this backing sheet in a manner similar to a sticker being removed from its backing sheet. A user then applies the dressing to the skin adhesive side down in the appropriate area. One skilled in the art will recognize that the preferred manner of adhering the dressing to the skin will be achieved when it is applied to dry skin free of hair. While it is possible to adhere the dressing to wet skin or to areas covered with hair, this situation may interfere with the effectiveness of the barrier as its effectiveness relies on securely adhering to one's skin.

The dressing further comprises a non-adhesive side coupled to a border sheet. This border sheet allows the dressing to maintain its shape and overall integrity after it is removed from the stiff backing sheet. Without the border sheet, the dressing would be extremely difficult to place appropriately on a body due to the thin, flexible nature of the dressing. The border sheet is anticipated to be a thin border around the non-adhesive side to the dressing. It is anticipated to be just wide enough to give the dressing shape and integrity during application. It is important that a user be able to see the dressing during application as any error in placing the dressing may cause the dressing to not lay flat and adhere properly against the skin. During application, the user will be able to see how the dressing is adhering to the skin and be able to correct for any misplacement, bubbles or folds in the dressing to ensure the appropriate placement and fit. After the dressing is adhered to the skin, the border sheet is to be removed.

The non-adhesive side of the dressing is preferred to have a smooth texture so as to not be obtrusive or diminish a user's sensations while wearing this dressing.

The dressing contoured to male anatomy may further comprise thigh/groin flaps or penile base collar. These flaps/collar are simply an extension of the dressing and are similarly coupled to a stiff sheet on their adhesive side and a border sheet on the non-adhesive side. The thigh/groin flaps simply provide for a more ergonomically fit around a user's thighs while adding some additional coverage for this area that may be affected by disease. The penile base collar adds a layer of barrier coverage to the penis as this area will be covered by an appropriately fit condom after the dressing is applied. The thigh/groin flaps and penile base collar may include a tab on the border sheet for ease of removal of the border sheet.

The dressing contouring to the female anatomy may include labial flaps. These flaps are simply an extension of the dressing and are similarly coupled to a stiff sheet on their adhesive side and a border sheet on the non-adhesive side. As the flaps are removed from the stiff backing sheet, they are then wrapped inwardly around the outer labia. The border sheet is removed after the flaps are appropriately placed. Tabs may be utilized for ease of border sheet removal.

This invention is anticipated to be used in conjunction with an appropriately sized condom. If a male partner is wearing the shield of this invention, then the condom is placed over the penis after the dressing is applied. If a female partner is wearing the shield of this invention, then it is anticipated that her male partner will wear a condom. While it is anticipated that only one partner, generally the affected partner, will wear the shield of this invention, one skilled in the art will recognize that both partners can wear the shield of this invention at the same time.

It is anticipated that the nature of this material will allow for extended wear of the shield. A user can apply the shield in advance of a sexual encounter and can continue to wear the shield after the sexual encounter. While it is preferred that the shield be changed in between sexual encounters, it can be worn for multiple encounters between the same partners. If sexual encounters between different partners are anticipated, then the shield must be removed and changed before an encounter with a new partner in order to reduce the risk of disease transmission.

The shield of this invention may be removed from the skin of a user by simply peeling the dressing away from the body. The dressing may then be thrown away.

This shield is anticipated to lay flat while in packaging prior to use.

It is anticipated that this shield will comprise a test patch so that a user can test for sensitivities to this this dressing. A user will be able to test in a small area of the body before applying it to a larger area.

This and other embodiments will be more thoroughly realized in the drawings and detailed description below.

### BRIEF DESCRIPTION OF THE SEVERAL VIEWS OF THE DRAWINGS

Possible and preferred features of the present invention will now be described with particular reference to the accompanying drawings. However, it is to be understood that the features illustrated in and described with reference to the drawings are not to be construed as limiting on the broad scope of the invention. In the drawings:

FIG. 1 is an rear exploded view of a shield contoured to fit the male anatomy in the arrangement preferred to fit the suprapubic region depicting the dressing, backing sheet, and border sheet.

FIG. 2 is front exploded view of a shield contoured to fit the male anatomy in the arrangement preferred to fit the genito-scrotal region depicting the dressing, backing sheet, and border sheet.

FIG. 3 is a perspective view of the shield contoured to the male anatomy, including a first and second barrier.

FIG. 3A is a detailed perspective view of the border sheet being removed by way of the tab.

FIG. 4 is a perspective view of the dressing contoured to the male anatomy, including a first and second barrier.

FIG. 4A is a is a perspective view of the dressing contoured to the male anatomy, including a first having an extended base adjacent to each side of the penile base collar but no thigh/groin flaps and second barrier.

FIG. 4B is a is a perspective view of the dressing contoured to the male anatomy, including a first having an extended base adjacent to each side of the penile base collar including thigh/groin flaps and second barrier.

FIG. 5 is a perspective view of the dressing as applied to the male anatomy including a shield over the subra-pubic region and a shield wrapped around the genito-scrotal region.

FIG. 6 is a cross-sectional view of the dressing as applied to the male anatomy.

FIG. 7 is an exploded view of the shield contoured to the female anatomy, depicting the dressing, backing sheet, and border sheet, including labial flaps.

FIG. 8 is a perspective view of the shield contoured to the female anatomy without labial flaps.

FIG. 9 is a perspective view of the shield contoured to the female anatomy including the labial flaps.

FIG. 10 is a perspective view of the dressing contoured to the female anatomy including the labial flaps.

FIG. 11 is a perspective view of the dressing as applied to the female anatomy.

FIG. 12 is a cross-sectional view of the dressing as applied to the female anatomy.

FIG. 12A is cross-sectional view of the shield depicting the labial flaps in use.

### DETAILED DESCRIPTION OF THE INVENTION

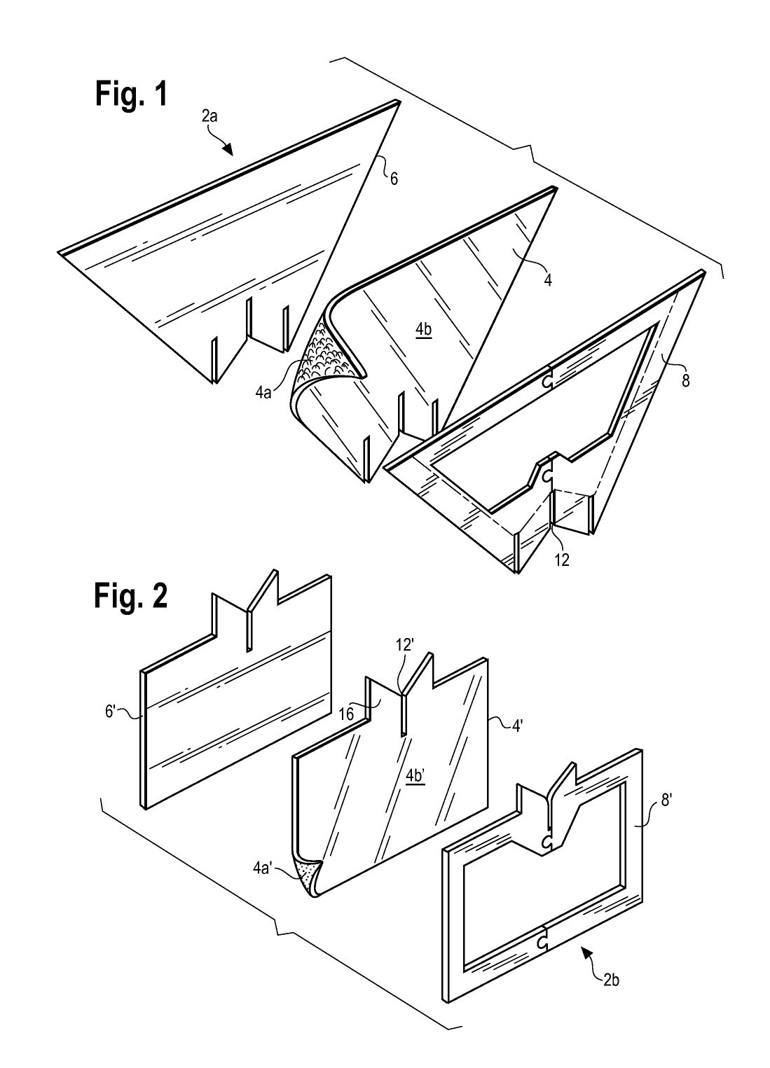

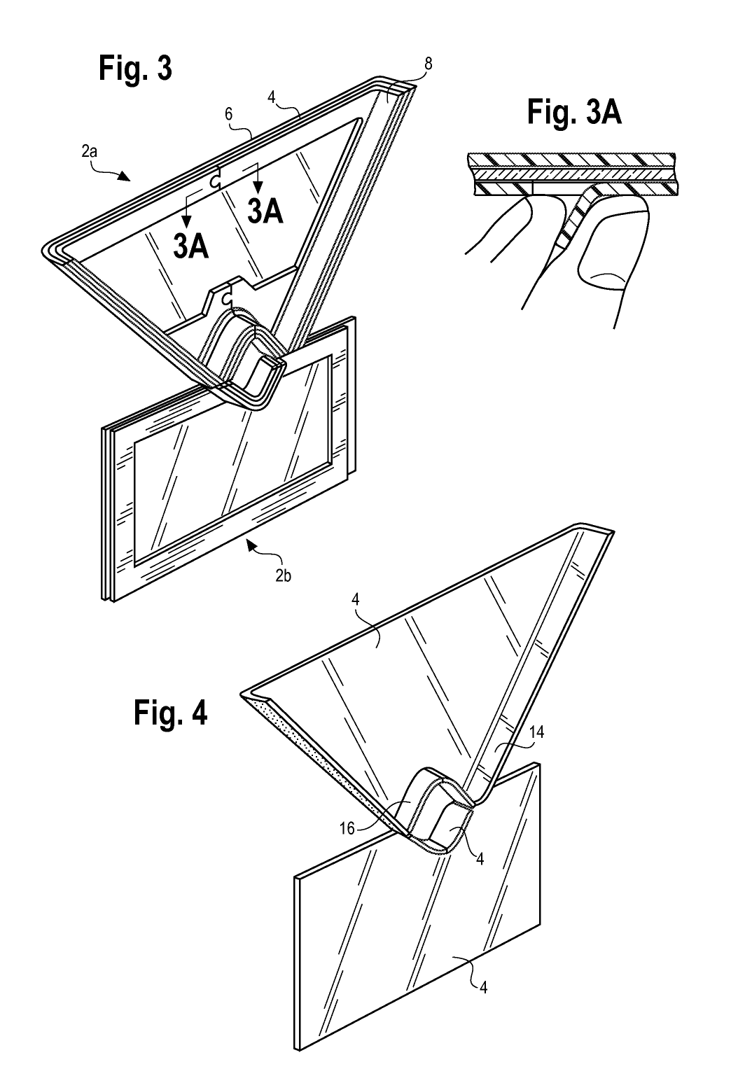

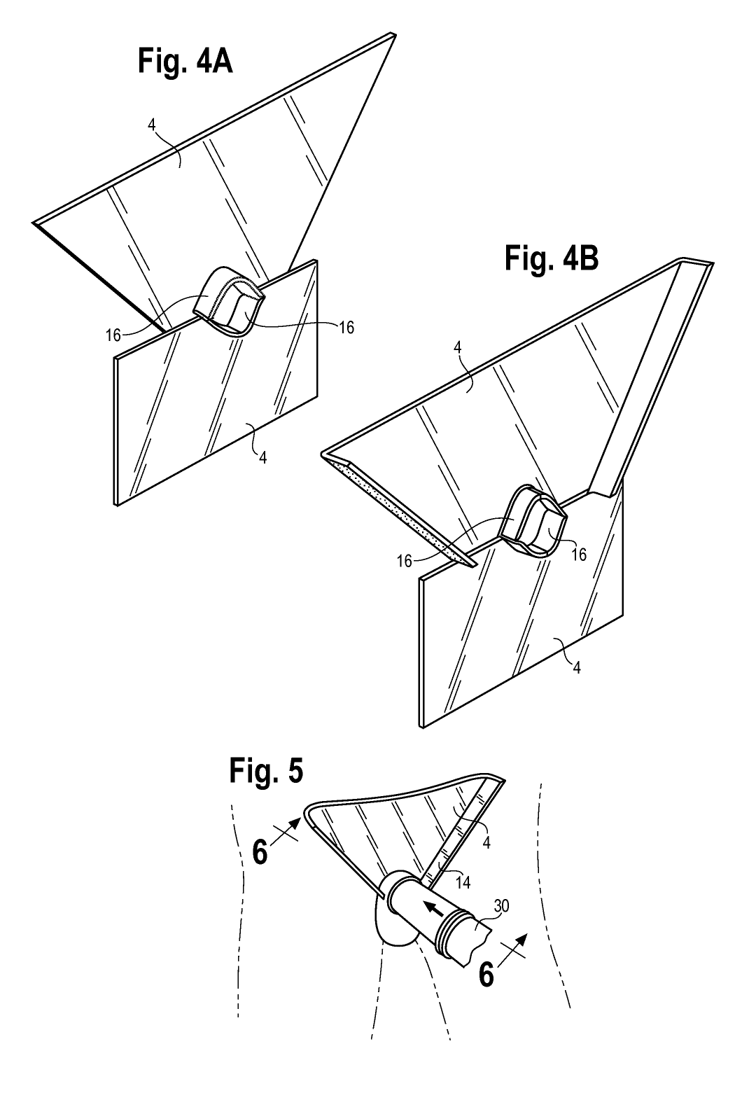

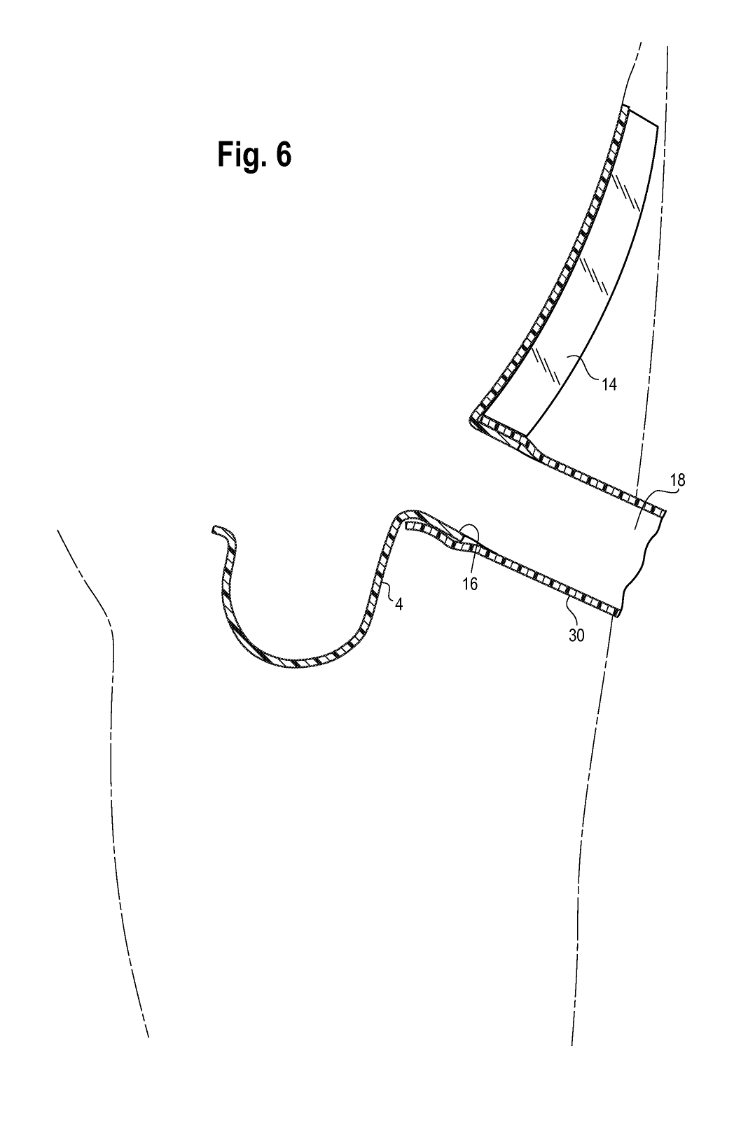

Turning to the Figures, FIGS. 1-6 relate to barriers 2a, 2b contoured to the male anatomy. FIGS. 1 and 2 depict an exploded view of the barrier 2a, 2b, including the backing sheet 6, dressing 4, and border sheet 8. Barrier 2a depicts a barrier contoured to fit male suprapubic region while barrier 2b depicts a barrier contoured to fit the male genito-scrotal region. Both barriers 2a and 2b include a penile base collar 16 while barrier 2a may also include thigh/groin flaps 14 as seen in FIG. 3. While only one barrier 2a or 2b may be used, for the most protection against disease, it is preferred that both barriers 2a and 2b be used together as seen in FIG. 3 or 4, 4A, 4B, 5, and 6.

The dressing 4 comprises an adhesive side 4a and a non-adhesive side 4b. The adhesive side 4a of the dressing 4 is joined to the backing sheet 6 and the non-adhesive side 4b is joined to the border sheet 8. Since it is anticipated that packaging for this barrier 2a, 2b will require the barrier 2 to lay flat when packaged, a slit 12 may be included on the penile base collar 16. The thigh flaps 14 will simply fold to lay flat while inside packaging.

As seen in FIGS. 3A, 5, and 6, to effectively use the barrier 2a contoured to the male anatomy, a user will peel the dressing 4 away from the backing sheet 6 and apply the adhesive side 4a to the user's skin. The penile base collar 14 wraps around the base of the penis 18 while the thigh flaps 14 are applied to the thighs. Once the dressing 4 is applied to the skin, the border sheet 8 can be peeled away exposing the non-adhesive side 4b of the dressing. A tab may be included (detailed in 3B) to assist in starting to peel the border sheet 8 away from the dressing. It is anticipated that a condom 30 will then cover the penis 18 and fit over the penile base collar 16. The dressing 2b is anticipated to wrap around and contour to the male scrotum.

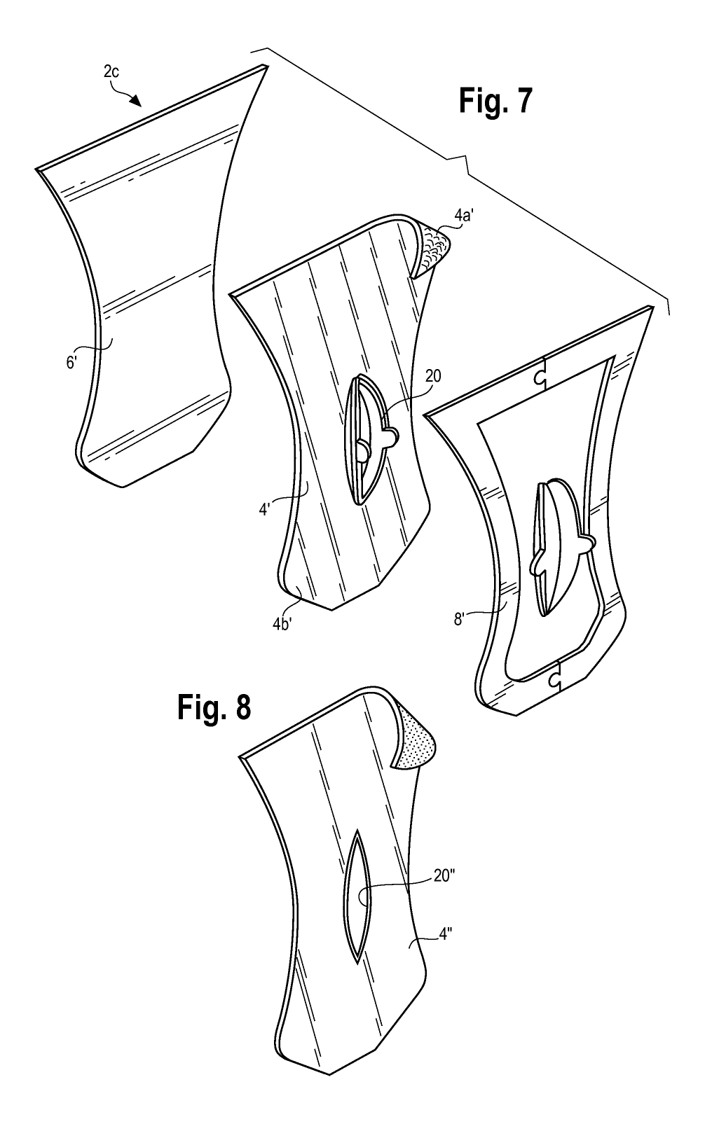

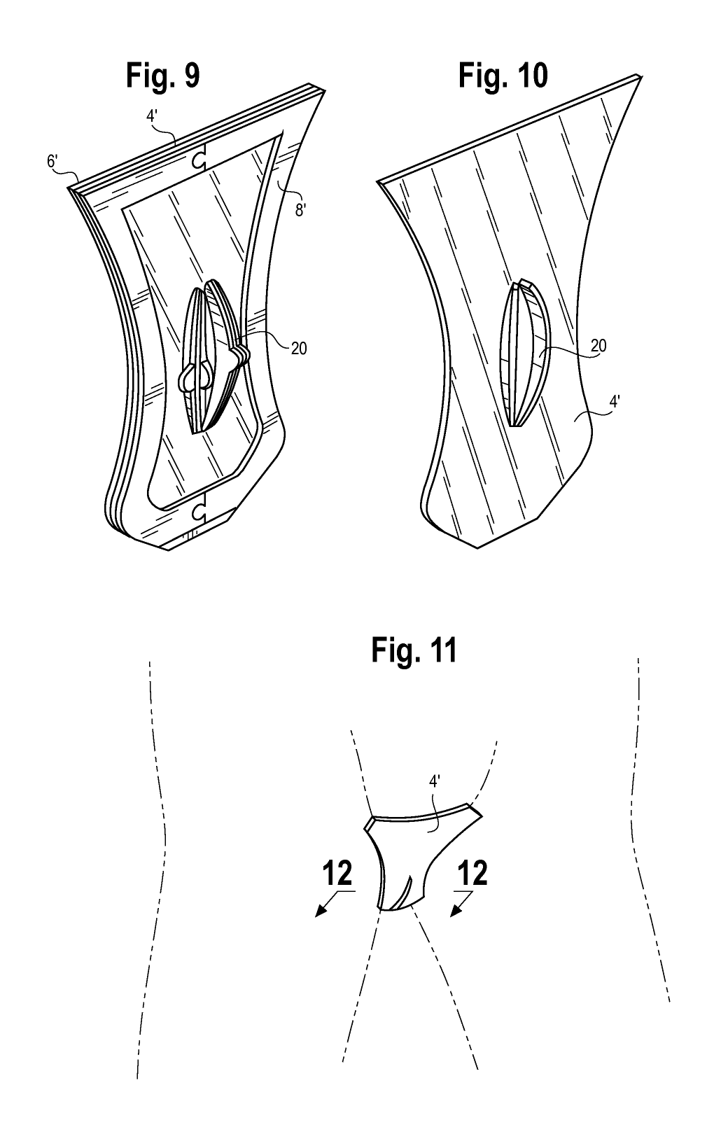

FIGS. 7-12A relate to a barriers 2c contoured to the female anatomy. FIG. 7 depicts an exploded view of the barrier 2a, including the backing sheet 6, dressing 4, and border sheet 8. FIG. 7 also depicts the barrier 2c including labial flaps 20. However, equally anticipated is the alternate arrangement seen in FIG. 8 without the labial flaps 20. The labial flaps 20 will fold to lay flat while inside of packaging before use.

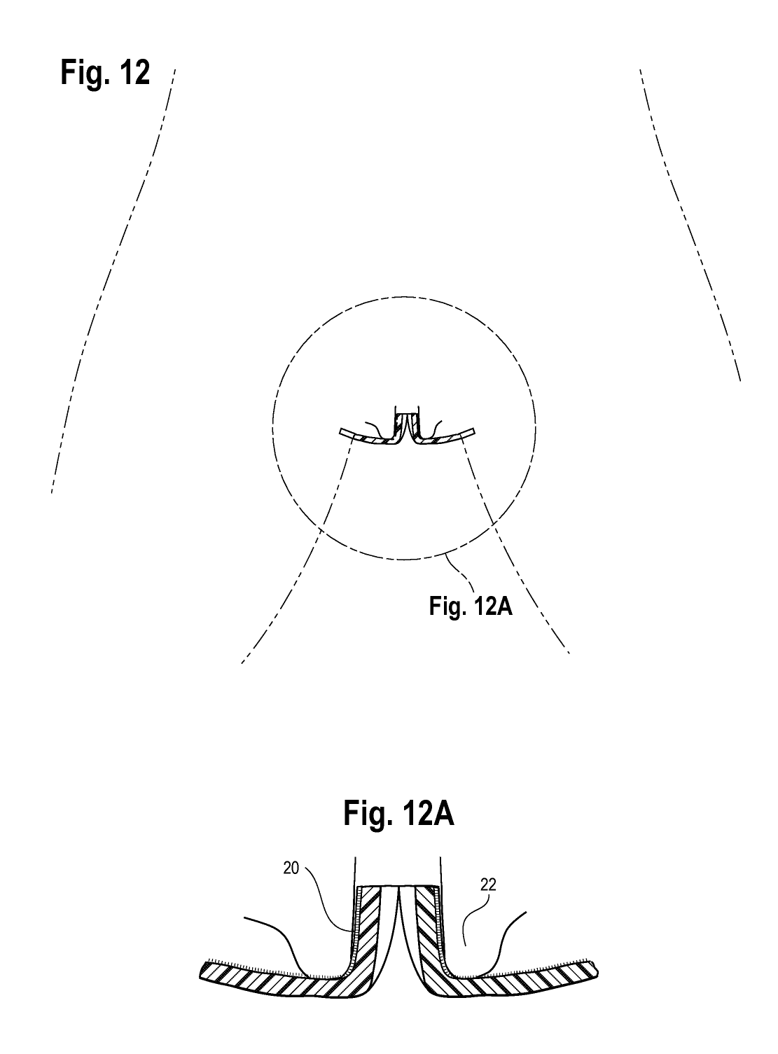

As in the male version, the dressing 4 of the female barrier 2c comprises an adhesive side 4a and a non-adhesive side 4b. The adhesive side 4a of the dressing 4 is joined to the backing sheet 6 and the non-adhesive side 4b is joined to the border sheet 8. To effectively use the barrier 2c contoured to the female anatomy; a user will peel the dressing 4 away from the backing sheet 6 and apply the adhesive side 4a to the user's skin. Once the dressing 4 is applied to the skin over the female labial and/or perineal region, the border sheet 8 can be peeled away exposing the non-adhesive side 4b of the dressing. In cases when labial flaps 20 are to be used, the dressing 4 will wrap inwardly around the skin of the labia 22 as seen in FIGS. 12 and 12A. The border sheet 8 will be removed after the labial flaps 20 are adhered appropriately to the labia 22. It is anticipated that for the most protection against disease, that the male partner of the female using this barrier 2c will also use a condom.

Throughout the specification and claims the word “comprise” and its derivatives are intended to have an inclusive rather than exclusive meaning unless the contrary is expressly stated or the context requires otherwise. That is, the word “comprise” and its derivatives will be taken to indicate the inclusion of not only the listed components, steps or features that it directly references, but also other components, steps or features not specifically listed, unless the contrary is expressly stated or the context requires otherwise.

It will be appreciated by those skilled in the art that many modifications and variations may be made to the methods of the invention described herein without departing from the spirit and scope of the invention.

What is claimed is:

1. A genital shield for application on a genital region for use in conjunction with a condom to aid in the reduction of the spread of sexually transmitted diseases comprising:
at least one barrier further comprising a dressing; said dressing having an adhesive side and non-adhesive side; said dressing is body shape contouring to male or female anatomy;
wherein the dressing is coupled to a backing sheet on the adhesive side and a border sheet on the non-adhesive side.

2. The shield as in claim 1, wherein the dressing comprises a material having a bio-occlusive nature.

3. The shield as in claim 1 wherein the dressing contouring to the female anatomy is applied by removing the dressing from the backing sheet and adhering the dressing to the skin of the female labial and perineal area; after adherence of dressing to the skin said border sheet is removed.

4. The shield as in claim 3, wherein the dressing contouring to the female anatomy further comprises labial flaps.

5. The shield as in claim 1, wherein the dressing contouring to male anatomy further comprises two barriers; wherein a first barrier is applied by removing the dressing from the backing sheet and adhering the dressing to the skin of the male genito-scrotal region and a second barrier is applied by removing the dressing from the backing sheet and adhering the dressing to the skin of the male suprapubic region; after adherence of the dressings to the skin said border sheet is removed.

6. The shield as in claim 5 wherein the first barrier further comprises thigh/groin flaps.

7. The shield as in claim 5 wherein the first barrier and second barrier further comprises a penile base collar.

8. The shield as in claim 5, wherein a condom is applied to a penis of a male after the barrier is applied.

9. A method for applying a genital shield to a genital area for use in conjunction with a condom to aid in the reduction of the spread of sexually transmitted disease comprising the step of:
applying at least one barrier to skin of the genital area; said barrier further comprising a body shape contouring dressing wherein said dressing comprises an adhesive side and non-adhesive side; said dressing contouring to male or female anatomy to the skin of the male or female; and wherein the dressing is coupled to a backing sheet on the adhesive side and a border sheet on the non-adhesive side.

10. The method as in claim 9 when applying the dressing contouring to the female anatomy further comprises the steps of:
removing the dressing from the backing sheet and adhering the dressing to the skin of the female labial area and
after adherence of dressing to the skin removing the border sheet.

11. The shield as in claim 10, wherein the dressing contouring to the female anatomy further comprises labial flaps and further comprises the step of adhering the labial flaps to the inside of the labia.

12. The method as in claim 9 when applying the dressing confirming to the male anatomy further comprising the steps of:
applying a first barrier by removing the dressing from the backing sheet and adhering the dressing to the skin of the male genito-scrotal region;
applying a second barrier by removing the dressing from the backing sheet and adhering the dressing to the skin of the male suprapubic region;
after adherence of the dressing to the skin removing the border sheet of the first barrier and second barrier; and
applying a condom to a penis.

13. The method as in claim 12 wherein the first barrier further comprises thigh flaps and further comprises the step of adhering the thigh/groin flaps to a thigh or groin area.

14. The method as in claim 12 wherein the first barrier and second barrier further comprising a penile base collar and further comprises the step of adhering the penis base collar to a penile base.
